(19) United States
(12) Patent Application Publication
(10) Pub. No.: US 2011/0126300 A1
(43) Pub. Date: May 26, 2011

(54) High Through-Put Method of Screening Compounds for Pharmacological Activity

(75) Inventor: Michael Pack, Philadelphia, PA (US)

(73) Assignee: The Trustees of the University of Pennsylvania

(21) Appl. No.: 12/086,948

(22) PCT Filed: Dec. 19, 2006
(86) PCT No.: PCT/US2006/048542

§ 371 Date: May 18, 2010

Related U.S. Application Data
(60) Provisional application No. 60/751,823, filed on Dec. 20, 2005.

(51) Int. Cl. A01K 67/00; C40B 30/06; C40B 40/02; A61K 31/55; C07D 487/04

(52) U.S. Cl. 800/8; 506/10; 506/14; 514/214.02; 540/578

(57) ABSTRACT

Provided is a method for high through-put screening for physiologic alterations in an altered teleost displaying a phenotype that is characteristic of the alteration and different from a wild-type, unaltered, matched teleost, comprising the steps of contacting the teleost displaying a genetically-inherited or chemically-induced phenotype with at least one test compound for a sufficient time and under suitable conditions to induce a response in the teleost indicative of pharmacological activity of the compound, and detecting and comparing the response with that of a matched, untreated, control, wherein a change in the teleost signal that is different from that of the control, indicates an altered phenotype and pharmacological activity of the at least one test compound. Further provided are the compounds identified by this method, the zebrafish having an altered phenotype resulting from treatment in accordance with these methods, and kits for facilitating the high through-put screening methods.

### REFERENCE TO RELATED APPLICATIONS

This patent application claims priority to Provisional Application 60/751,823, filed Dec. 20, 2005 and PCT Application PCT/US2006/048542, filed Dec. 19, 2006, which is herein incorporated in its entirety.

### GOVERNMENT SUPPORT

This work was supported in part by National Institutes of Health Grants DK54942 and DK61142 and core facilities and training grants provided by an NIH Center Grant (P30) DK50306. As a result, the government may have certain rights to this invention.

### BACKGROUND OF THE INVENTION

The traditional approach to drug discovery is to identify target genes involved in a disease and then design an in vitro assay to screen small molecules to determine which ones cause changes in the function of the target. However, such a traditional approach is flawed, not only because of its high cost and inefficacies in identifying target genes and because limited animal models are available, but because the protein configurations used in most pharmaceutical industry assay systems are radically different from that which is found in vivo. The protein in such in vitro assays is typically in crystalline form, in an aqueous solution, and attached to a fixed bed or overexpressed in a transfected cell. Thus, the inefficiencies and costs associated with traditional approaches to drug discovery and the difficulties associated with handling proteins in vitro, have made is necessary to develop an experimental vertebrate model system which is less costly and more efficient, in which the targets are found in native configurations. Unfortunately, however, many current assays regarding angiogenesis, cancer, and the like do not permit in vivo assessment of compounds or their side effects in a whole animal model, or in multiple tissues or organs of animal models over time. In addition, many current assays for new drug activity are not suitable for rapid, automated, or extensive compound screening, particularly screening of compound libraries containing numerous complex compounds.

There are currently two approaches for detecting metabolic activity in vertebrate hosts. The first approach uses standard cell culture techniques and typically relies on standard microplate readers to detect the death of cells cultured from an organism. A major drawback of the cell culture assay format is that it does not permit analysis of the effects of a compound on cell types that have not been cultured (i.e., other cell types). It also does not allow evaluation of the effects of a compound on specific tissues or organs or in an intact whole host in vivo. Furthermore, such assays do not permit monitoring of cellular activity in multiple tissues, organs, or systems simultaneously or over time in a live host. As a result, the cell culture assay approach does not allow for rapid or automated high-throughput screening of many compounds.

A second approach utilizes a histochemical staining technique, designated terminal deoxyuridine nucleotide end labeling (TUNEL), to detect dead or dying cells in sectioned tissues of vertebrate embryos. Unfortunately, with this approach, only a single time point can be examined. The changes in various tissues or organs of the subject over time cannot be monitored. Because many degenerative diseases occur in stages, the ability to examine changes in activity caused by a compound and the duration of direct and side effects of the compound on multiple tissues and organs would represent a significant improvement over such methods. Moreover, because the TUNEL approach requires that cells be fixed for visualization, effects in a live animal cannot be monitored.

In addition, current screens do not permit the assessment of drug effects on all potential target cells, tissues, or organs of an animal; nor can the effects of a compound on multiple target tissues and organs be currently assayed simultaneously or over time. Also, some potential therapeutic compounds, although they do not produce immediate lethality, do induce toxic effects in specific organs and tissues. Consequently, many compounds that pass preliminary cell-based testing, fail final large animal toxicity testing, which is a prerequisite for eventual FDA approval, demonstrating that more predictive and comprehensive toxicity screening methods are needed in whole animals and in one or more designated target tissues or organs in vivo (and in cells in vitro) over time than are currently available.

By comparison, studies have shown that intestinal anatomy and architecture in cyprinid teleost fish is closely related to mammals (Curry, J. Morphol. 65:53-78 (1937); Rogick, J. Morphol. 52:1-25 (1931)), and Wallace et al., Mechanisms of Development, 122:157-173 (2005a) showed by histological and immunohistochemical analyses that the features are conserved in zebrafish. In particular, their post-mid-blastula transition (MBT) embryos have been shown to be highly representative of responses in other vertebrates (Ikegami et al., Zygote 5:329-350 (1997)). Because many aspects of zebrafish organ physiology have been conserved during vertebrate evolution, genetic screening to assay organ function in the optically transparent zebrafish is a valuable approach to understanding a variety of metabolic processes and disorders in vertebrates. Zebrafish have an extremely rapid embryonic development (3 days) and short maturation period (2-3 months). As a result, it is relatively simple to generate large numbers of the embryos and larvae. Zebrafish embryos and larvae are relatively large and translucent, and begin dividing synchronously and rapidly (approximately 15 min cell cycles) until MBT, which occurs after about 10 cell divisions. In many ways zebrafish anatomy and physiology is comparable to mammalian, and their external fertilization and extracorporeal development has made them a model of choice for transgenic research (Stuart et al., Development 109:577-584 (1990); Culp et al., Proc. Natl. Acad. Sci. USA 88:7953-7957 (1991); Hammerschmidt et al., Methods Cell Biol. 59:87-115 (1999)).

Four day larval zebrafish intestines evidence visible peristaltic contractions, and are microscopically differentiated, with a polarized epithelium bearing the characteristic absorptive, endocrine, and goblet cells of higher vertebrates. The interaction between epithelial cells and mesenchymal cells in the intestine is essential for intestinal organ development. During development of the digestive system, the signals from mesenchymal cells direct architectural patterns in epithelial cells, thus forming the normal gut (Kedinger et al., Ann. N.Y. Acad. Sci. 859:1-17 (1998); Roberts et al., Development 125:2791-2801 (1998); Kaestner et al. Genes Dev. 11:1583-1595 (1997; Karlsson et al., Development 127:3457-3466 (2000); Pabst et al., Development 126:2215-2225 (1999)); Wallace et al., supra, 2005a).

At the anterior end of the gastrointestinal tract is an esophagus and at the posterior is a short region believed to be a homologue of the colon. One bud off the gut forms the liver, composed of cords of hepatocytes and bile ducts, and another, the pancreas, with an insulin-generating islet surrounded by exocrine cells. Conversely, the epithelial cells signal the mesenchymal cells to differentiate into smooth muscle cells (Haffen et al. J. Pediatr. Gastroenterol. Nutr. 6:14-223 (1987). The necessity of this interplay is highlighted in disease. Adenomatous neoplasia can occur when BMP signaling from the stroma is disrupted (Haramis et al., Science 303:1684-1686 (2004)). Furthermore, laboratory studies with targeted disruption of TGF-β receptors can lead to stomach tumors, confirming the necessity for normal mesenchymal-epithelial interaction (Bhowmick et al., Science 303:848-851 (2004)).

At least nine recessive lethal mutations that perturb development of the digestive organs have already been identified by zebrafish chemical mutagenesis screening (Drieve et al., Development 123:37-46 (1996); Pack et al., Development 123:321-328 (1996)). Although the mutants were identified using morphological criteria, their phenotypic analysis suggests that in some cases the affected genes regulate developmental processes that are relevant to digestive physiology and other aspects of vertebrate metabolism. The recessive lethal meltdown (mlt) mutation causes cystic expansion of the larval zebrafish posterior intestine (Pack et al., 1996, supra). Epithelia of the anterior intestine and other organs are unaffected in mlt mutants and heterozygous mlt larvae develop normally. Mutant strains of zebrafish have been used as models for the study of human blood disorders, such as congenital sideroblastic anemia (Brownlie et al., Nat. Genet. 20:244-250 (1998)) and hepatoerythropoietic porphyria (Wang et al., Nat. Genet. 20:239-243(1998); reviewed in Bahary et al., Stem Cells 16:89-98 (1998); Amatruda et al., Dev. Biol. 216:1-15 (1999)).

However, limitations have become apparent that are inherent to genetic screens based solely on morphological criteria in the zebrafish analyses. Despite the transparency of the zebrafish larva, the function of few organs can be effectively assayed by visual inspection alone. Not all organs are readily distinguished in the embryos or larvae, and mutations that perturb organ morphology are often overlooked. Moreover, since it is difficult to visualize specific cell populations within many larval organs, mutations that affect the development or function of these cells can be overlooked. Consequently, morphology-based screens are better suited for the identification of genes that regulate specification and patterning of embryonic structures, whereas by contrast, biomedical screens are most effective when they directly assay physiological processes.

Because of inefficacies and costs associated with the traditional approaches to drug discovery and difficulties associated with handling proteins in vitro, there has remained an unmet need to provide novel, medically-relevant high through-put screening assays and methods for assaying physiological processes in an animal model for many human diseases, including cancer, inflammatory and cardiovascular disease, and congenital and acquired diseases of the intestine and liver. Such assays are preferably amenable to large scale screening to identify biologically active small molecule compounds, as well as previously unknown activities for known compounds, for the treatment of human disease and/or other medical conditions.

### SUMMARY OF THE INVENTION

The present invention provides assays and methods for high through-put screening for physiologic processes in an altered teleost displaying a phenotype that is characteristic of the alteration and different from a wild-type, unaltered, matched teleost. The method comprises contacting the a teleost displaying a genetically inherited or chemically induced altered phenotype, with at least one test compound for a sufficient time and under suitable conditions to induce a response in the teleost indicative of pharmacological activity of the compound; introducing a labeled reagent to the contacted teleost and test compound under conditions that allow for uptake of the reagent by the teleost, wherein binding of the labeled reagent to, or with, the at least one teleost generates a detectable signal dependent upon and characteristic of the teleost's response; and detecting the signal and comparing it to the response from a matched control teleost that was not contacted with the test compound or the labeled reagent, wherein a change in the teleost signal that is different from that of the control, indicates an altered phenotype and pharmacological activity of the at least one test compound. The embodied teleost is an embryonic, larval or adult zebrafish.

It is an object of the invention to provide a high through-put screening method, wherein the altered phenotype is associated with and representative of a disease, such as cancer, hematologic disease, immunologic disease, angiogenesis, rheumatoid arthritis, atherosclerosis, cardiovascular disease, obesity and cholesterol deposits, mellitus, retinopathies, psoriasis, bone diseases, liver diseases, and retrolental fibroplasias, neurodegenerative disease and metabolic disorders, or wherein the phenotype is useful for studying metabolic processes.

It is also an object of the invention to provide a method for high through-put screening of a test compound for the ability alter a genetically or chemically altered teleost displaying a phenotype that is characteristic of the alteration and different from a wild-type, unaltered, matched teleost. The method comprises the steps of contacting the teleost displaying a genetically inherited or chemically-induced phenotype, with at least one test compound for a sufficient time and under suitable conditions to induce a response in the teleost indicative of pharmacological activity of the compound; introducing a labeled reagent to the contacted teleost and test compound under conditions that allow for uptake of the reagent by the teleost, wherein binding of the labeled reagent to, or with, the teleost generates a detectable signal dependent upon and characteristic of the teleost's response; and detecting the signal and comparing it to the response from a matched control teleost that was not contacted with the test compound or the labeled reagent. A change in the teleost signal that is different from that of the control, indicates an altered phenotype and pharmacological activity of at least one test compound.

It is a further object to provide a compound obtained by the methods of the present invention. And it is an additional object to provide zebrafish embryos, larva or adults, having an altered phenotype resulting from treatment in accordance with the present methods, wherein the alteration indicates activity of the test compound.

In addition, it is an object to provide methods for treating a host having, or susceptible to, a disease or disorder characterized by uncontrolled cellular invasion or fibrosis, said method comprising administering a test compound selected by the present methods, wherein the labeled reagent is pharmaceutically acceptable. Such methods may further comprise identifying a gene(s) involved in the regulation of cellular invasion, in particular involving cancer or fibrosis.

Moreover, it is an object of the invention to provide a kit comprising packaging material and the necessary teleosts, together with a pharmaceutically acceptable marker, wherein said packaging material comprises a label which indicates uses of the contents of the kit for high through-put screening for a composition causing physiologic alterations in an altered teleost displaying a phenotype that is characteristic of the alteration and different from a wild-type, unaltered, matched teleost.

Additional objects, advantages and novel features of the invention will be set forth in part in the description, examples and figures which follow, and in part will become apparent to those skilled in the art on examination of the following, or may be learned by practice of the invention.

### BRIEF DESCRIPTION OF THE FIGURES

The foregoing summary, as well as the following detailed description of the invention, will be better understood when read in conjunction with the appended figures, which are not intended to be limiting.

FIGS. 1A-1D photographically show the effects of an automated suppressor screen. FIG. 1A shows wild type and 1B shows mlt larvae that have ingested PED6 quenched fluorescent lipid. FIGS. 1C and 1D are enlargements of FIGS. 1A and 1B, respectively.

FIGS. 2A-2D photographically show lateral fluorescent images of 6 dpf zebrafish larvae following 2 hour exposure to fluorescent lipid PED6. As shown in FIG. 2A, the anterior and posterior wild type intestine contains the PED6 lipid (black arrowheads), whereas the mlt intestine in FIG. 2 C shows little PED6 in the distal posterior intestine (open arrowhead). Wild type larvae exposed to SB432542 (FIG. 2B) show a normal intestine contour, whereas the mlt Larvae exposed to SB431542 (FIG. 2D) show posterior intestinal fluorescence and the contour is normal as a result of inhibition of the cyst formation process.

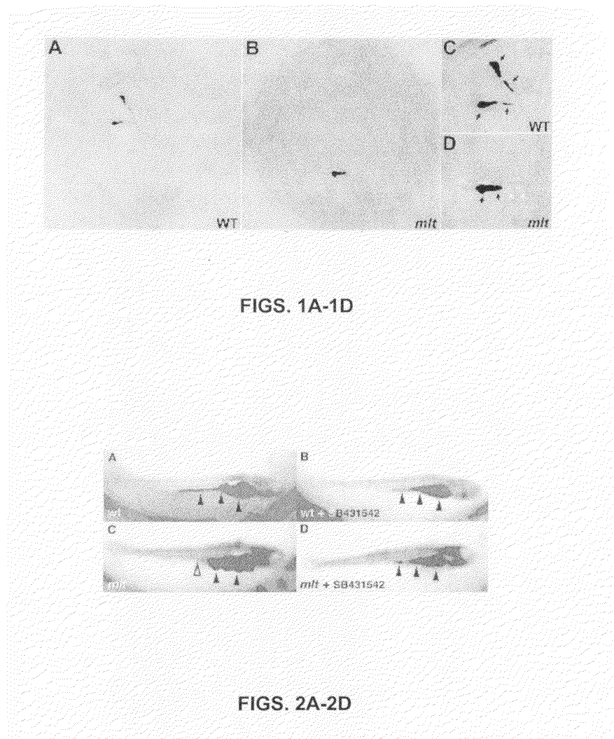

### DETAILED DESCRIPTION OF CERTAIN PREFERRED EMBODIMENTS

Within the past few years, the discovery and analysis of zebrafish mutants affecting organogenesis has confirmed an important role for the zebrafish in biomedical research. Given the shared features of mammals and teleosts, zebrafish mutagenesis screens using lipid reporters can be used to identify genes with functions relevant to human diseases, including cancer, inflammatory and cardiovascular disease, and congenital and acquired diseases of the intestine and other organs. The present invention involves utilizing fluorescent lipids to screen for the rescued phenotypes representing physiological perturbations arising from mutations of specific genes that lead to disorders affecting non-metabolic functions, e.g., cancer cellular invasion, such as cancer cell invasion, or organ fibrosis. The ability to apply high through-put genetic analyses to vertebrate organ physiology using this model system is unprecedented and will complement research in other vertebrate model systems, including but not limited to, rodents, amphibia, and fish.

The present invention teaches a novel, target-blind approach to drug discovery. Model human phenotypes, e.g., disease phenotypes, are provided in a teleost, such as a zebrafish, and then compounds, e.g., small molecules, are screened for their ability to alter the phenotype. Because the screen is performed in a whole vertebrate organism and uses a phenotype as the output, the need to first identify target genes is eliminated. Thus, a single screen may theoretically detect, for example, drugs affecting any target relevant to a disease phenotype being observed, even if those targets are not yet characterized.

In general, the present invention provides a method of screening a test compound for the ability of the compound to alter a phenotype, preferably modeling a human phenotype. The method comprises the steps of (1) contacting at least one teleost that has inherited the phenotype with a test compound, and (2) detecting phenotypic alterations in the teleost from the first step. By conducting a mutagenesis screen using fluorescent lipids, which would not be feasible with standard zebrafish screening strategies, the power of high through-put genetic analysis can be used to identify drugs or other compositions that inhibit non-metabolic functions, e.g., cancer cellular invasion, such as cancer cell invasion, or organ fibrosis, that have important implications for human diseases.

The fluorescent phospholipids PED6 [N-((6-(2,4-dinitrophenyl)amino)hexanoyl)-1-palmitoyl-2-BODIPY-FL-pentanoyl-sn-glycero-3-phosphoethanolamine (PED6)], described in the present invention, was used with a phospholipase PLA₂ developed by Hendrickson et al. Anal Biochem. 276(1):27-35 (1999) to analyze phenotypic changes in the intestinal and hepatobiliary system of teleosts. For example, the reagents have been used to examine cholesterol synthesis (Faber et al., Science 292(5520):1385-1388 (2001); US Publ. Pat. Appl. 2003/0135869; 2002/0162124 and 2002/0049986 and the detailed description of the reagents used therein, which are herein incorporated by reference). In that study the fluorescently quenched phospholipids were ingested by the larvae and endogenous lipase activity and rapid transport of cleavage products resulted in intense gall bladder fluorescence, permitting the identification of zebrafish mutants, such as fat free, that show normal digestive organ morphology, but have severely reduced phospholipid and cholesterol processing.

In the present invention, however, these fluorescently-tagged reagents become a powerful tool for identifying genes that mediate a wide range of vertebrate digestive developmental and physiological processes including, but not limited to, swallowing, digestion, absorption, and transport; esophageal sphincter function; intestinal motility; organogenesis of the mouth and pharynx, esophagus, intestine, liver, gallbladder and biliary system, and exocrine pancreas and ducts; and the cellular and molecular biology of PLA₂ regulation, cell invasion, organ fibrosis, polarized transport, and secretion.

The Teleost Model

As used herein, the term “teleost” means a vertebrate of or belonging to the Teleostei or Teleostomi, a group consisting of numerous fishes having bony skeletons and rayed fins. Teleosts include, for example, zebrafish (Danio rerio), Medaka, Giant rerio, and puffer fish. In an embodiment of the invention, the teleost is a zebrafish. However, while zebrafish are described herein in the exemplified embodiments, the invention need not be so limited. The teleosts used herein are wild-type and mutants. The teleost may be in any stage of its life-cycle, including embryo, larva or adult. In certain preferred embodiments, the teleost is a zebrafish embryo or larva. Mutant embryos and larvae are selected with particular phenotypes, or in the alternative mutant or wild type embryos or larvae are modified in some way to facilitate high through-put screening or they are transgenic embryos or larvae with a particular phenotype or organ-specific visible marker. Larvae are particularly useful for the present methods. Mutant strains of teleosts (such as zebrafish) may be used to assess, e.g., the interaction between therapeutic agents and specific genetic deficiencies. The teleost may contain mutations in a selected gene, such as a heritable mutation, including, e.g., a heritable mutation associated with a developmental defect. The teleost can also be transgenic, or the teleost may be otherwise normal, until treated with a chemical that induces a disease state, such as a chemical compound that induces seizures in a zebrafish larva, thereby permitting testing in those larva to find active agents that may ameliorate or prevent seizures in a human or in a representative animal.

Zebrafish provide a relatively simple model system for more complex vertebrates, such as humans. They are small in size, easy to maintain and breed, and produce large numbers of progeny on a daily basis. Their embryos develop rapidly and are optically clear, permitting direct observation of the developing digestive system. Because they are vertebrates, zebrafish contain orthologues for almost all human genes. The species also is amenable to genetic methods so that one can screen for mutations that disrupt organ function or development. It is possible, therefore, to identify genes important for intestinal development and function by examining mutant fish that display phenotypic changes. In addition, many techniques are well known for manipulating zebrafish, including in vitro fertilization, production of haploids and parthenogenic diploid embryos, mutagenesis, cell lineage and cell transplantation.

Unless otherwise defined, all technical and scientific terms used herein have the same meaning as commonly understood by one of ordinary skill in the art. See, e.g., Singleton et al., Dictionary of Microbiology and Molecular Biology (2d ed. 1994); The Cambridge Dictionary of Science and Technology (Walker ed., 1988); and Hale & Marham, The Harper Collins Dictionary of Biology (1991). Typically, animal models of the present invention are optically clear in at least one of the following stages: embryo, larva, or adult. The “embryo” is a recognized stage following embryogenesis, before the larval stage. The term “larva” or “larval” as used herein means the stage of any of various animals, including vertebrate animals (including teleosts, e.g., zebrafish, etc.), between embryogenesis and adult.

In general, the body plan, organs, tissues, and other systems of teleosts develop much more rapidly than do such components in other vertebrate model systems (e.g., the mouse). The entire vertebrate body plan of the zebrafish, for example, is typically established within 24 hours. A functioning cardiovascular system is evident in the zebrafish within the first 24 hours of development (Stainier et al., Trends Cardiovasc. Med. 4:207-212 (1994)). The remaining organs of the zebrafish, including the gut, liver, kidney, and vasculature, are established within the ensuing 48 hours. The hatched zebrafish embryo nearly completes morphogenesis within 120 hours, thereby making it highly accessible to manipulation and observation. At the end of embryogenesis, all of the major vertebrate organs are represented.

Compounds permeate the intact larvae (or embryos) directly, making culture of the teleosts in a multi-well format particularly attractive for high through-put and automated compound screening. Advantageously, both the therapeutic activity and side effects (e.g., toxicity) of a drug can be assayed simultaneously in vivo in teleost model systems. Automated imaging enhances assay chemical efficacy.

By “alter,” “altering,” “alteration” and the like is meant a change or modulation of the inherited normal phenotype of a teleost, or the expected phenotype of a teleost mutant. A chemical compound alters the phenotype when the statistically expected pattern of phenotype inheritance produces fewer mutants than expected in the presence of a test compound. For example, an alteration may be detected in teleost embryos, wherein the embryos are produced by mating heterozygous zebrafish, wherein each has a lethal recessive phenotype. The resulting embryos (or larva) are consequently contacted with a test compound, as explained in detail in the examples below, and visually examined, for example, for increased or decreased staining under a light microscope, using bright field or fluorescent imaging. In some methods, the detectable signal is an optically detectable signal, which can be detected, for example, by a microplate reader.

Small molecule test compounds typically penetrate the teleost embryos by simple diffusion. For compounds that do not penetrate the periderm (the outer ectoderm), dimethyl sulfoxide (DMSO), or other solvents, or osmotic shock can be used to transiently permeabilize the periderm. Compounds can also be administered by other well-known methods of administration, including ingestion or direct injection into either the embryo yolk or the heart of the teleost embryo. Once inside the embryo, compounds diffuse freely within the embryo.

The visual marker may be provided to the teleost using a dye associated with an activity of, or by, the test compound (e.g., cell death activity, angiogenesis activity, toxic activity). Dyes can be administered alone, in conjunction with a variety of solvents (e.g., dimethylsulfoxide (DMSO) or the like), or in conjunction with other dyes or markers. Such secondary dyes or marker may be administered to the teleost before, at the same time as, or after administration of a dye used for detection of the response in the teleost indicating a specific activity. In the methods of the Examples that follow, a fluorescent marker, phospholipid PED6, is disclosed, but other markers are also effective. PED6 fluorescent metabolites enhance visibility of digestive organ structure, facilitating scoring of gall bladder development, intestinal folding, differentiation motility and architecture and bile duct development (Farber et al., 2001, supra).

Fluorescent Reagents to Assess in Vivo Organ Physiology

The fluorescent lipids of the instant invention allow assaying of physiological processes. The reagents are fluorescent analogues of compounds that could serve as modifiable substrates in important metabolic and signaling pathways. The reagents of the instant invention were constructed by covalently linking fluorescent moieties to sites adjoining the cleavage site of phospholipids. Dye-dye or dye-quencher interactions modify fluorescence without impeding enzyme-substrate interaction (Hendrickson, Anal. Biochem. 219:1106 (1994)). PLA₂ cleavage results in immediate unquenched and detectable fluorescence. The quenched phospholipid [N-((6-(2,4-dinitrophenyl)amino)hexanoyl)-1-palmitoyl-2-BODIPY-FL-pentanoyl-sn-glycero-3-phosphoethanolamine (PED6)] allows subcellular visualization of PLA₂ activity and reveals organ-specific activity (Farber et al., J. Biol. Chem. 274:19338-19346 (1999)). As shown by Farber et al., zebrafish larvae 5 days post-fertilization (dpf) bathed in PED6 show intense gall bladder fluorescence and, shortly thereafter, intestinal luminal fluorescence. Substrate modification allows localization of enzymatic activity by altering the emission spectrum of the fluorescent compounds. When used in the context of a genetic screen, these fluorescent lipids provide a high through-put readout of organ function.

The reagents of the present invention facilitated the development of genetic screens that are more sensitive than the whole-mount in situ and antibody based screening protocols now used to assay gene expression. The fluorescent reagents are simpler to use since they can be administered to and assayed in a wide range of organisms, including, but not limited to rodents and teleosts, and they offer the opportunity to screen for hypomorphic mutations that alter gene function, but do not affect levels of gene expression. By providing a visual assay of metabolic or physiological processes, these reagents can be used to identify mutations that affect more than just the single gene responsible for substrate modification. Visualization of the fluorescent signal also is dependent upon the delivery and uptake of the substrate as well as the storage, metabolism or secretion of its modified metabolites. A target enzyme, such as a phospholipase is not needed for the reagent of the present invention, such as PED6, because the fluorescence is used herein to demonstrate organ development and the presence of cellular invasion, which is turned on or off by the presence of the digestive track mutation, such as mlt, rather than by an enzymatic or cell cycle activity.

The fluorescence emission of the dye markers is monitored using standard fluorometric techniques, including visual inspection, CCD cameras, video cameras, photographic film, or the use of current instrumentation, such as laser scanning devices, fluorimeters, photodiodes, quantum counters, photon counters, plate readers, epifluorescence microscopes, scanning microscopes, confocal microscopes, or by means for amplifying the signal, such as a photomultiplier tube. Such dyes are generally discussed in U.S. Pat. No. 5,658,751, herein incorporated by reference. A number of suitable fluorescent dyes are commercially available.

Dyes can be selected to have emission bands that match commercially available filter sets, such as those used for detecting fluorescein or multiple fluorophores with several excitation and emission bands. Another factor to consider is the toxicity of the dye. The use of non-toxic dyes permits monitored of the cells over a significant time period, without risk that the teleost will be adversely affected by the dye. By comparison, assays (e.g., TUNEL labeling) using other types of markers require that the host be sacrificed and fixed. As a result, dyes such as fluorescent phospholipid PED6, are particularly suitable markers for high through-put, automated screening methods.

In alternative methods, a labeling reagent or marker is a substrate of an enzyme, and the response is an increase or decrease in activity of the enzyme. In other methods, the labeling reagent or marker comprises an antibody, and the detectable signal is generated by the antibody bound to a cellular receptor of the teleost. Yet additional methods provide a response as a change in number of cells or types of cells or morphology of the teleost.

In certain other embodiments, the labeling reagent is a nucleic acid, and the detectable signal is generated by the nucleic acid bound to a nucleic acid of the teleost. In other methods, the labeling reagent is contacted with a second labeling reagent that binds to the labeling reagent, thereby generating the detectable signal. In some methods, the pharmacological activity is the modulation of angiogenesis, organ morphology/architecture or cancer, e.g., for angiogenesis the response may be, but is not limited to, a change of alkaline phosphatase activity of the teleost.

Screening

Teleosts, including zebrafish, offer important advantages over other animal model systems for use in screening methods of the present invention. First, teleosts are vertebrates, meaning that their genetic makeup is more closely related to that of man as compared with other model systems in Drosophilae and nematodes. All essential components of human form and organ development are mimicked in these teleosts and the morphological and molecular bases of tissue and organ development are either identical or similar to other vertebrates, including man (Chen et al., Development 123:293-302 (1996); Granato et al., Cur. Op. Gen. Dev. 6:461-468 (1996)). As a result, teleosts serve as an excellent model for the study of vertebrate development and human disease states.

Secondly, teleosts are advantageous animal models because their embryos are highly transparent, meaning that angiogenesis activity, cell death activity (e.g., apoptosis and necrosis), and toxic activity produced by administered test compounds can be detected and diagnosed much more rapidly than in non-transparent animals. While these activities can also be detected in the more mature larval and adult forms of the zebrafish, observation is more difficult as such forms become progressively less optically clear. Nevertheless, the activities can be detected in vivo in all three forms, or in cells selected therefrom in vitro. As compared with the teleost embryos, other recognized animal models, such as the mouse embryo, for example, that develop in utero, must be removed from the mother by labor intensive procedures, before an assay can be performed or observed.

Test compounds can be administered directly to the developing teleost, whereas direct introduction of candidate test compounds is hindered in animals which develop in utero. Further, the teleost embryo is an intact, self-sustaining organism, whereas by comparison mammalian model animals when physically removed from its mother's womb, is not self-sustaining or intact. Additionally, single whole teleost embryos can be maintained in vivo in fluid volumes as small as 50 μl for the first six days of development, and intact live embryos can be kept in culture in individual microtiter wells or multi-well plates. As a result teleosts provide significant advantages in terms of not only the testing process, but also of time, space and cost over other high through-put assay systems.

Usually some wells of the multi-well plate are occupied by positive and/or negative controls. Positive controls comprise agents(s) known to have the pharmacological activity being tested, whereas negative controls comprise agent(s) known to lack the pharmacological activity. In certain embodied methods, multiple positive and/or negative controls are distributed at different locations on the plate.

Assay scoring may be done manually, or by automated means using devices known in the art for such purposes, using either commercially available or modified software programs or de novo software programming designed specifically for such assays. Secondary screens with compounds that are chemically related to the active compounds identified in the primary screen.

The term “test compound” as used herein, comprises any element, compound, or entity, including, but not limited to, e.g., a pharmaceutical, therapeutic, pharmacologic, or holistic medicament; an environmental or an agricultural pollutant or compound; an aquatic pollutant; a cosmetic product; a drug; or a toxin. Such test compound comprises natural or synthetic compounds, or a chemical compound or a mixture thereof which may be mixed with, or alternatively, dissolved in an aqueous mixture. The test compound may further comprise nucleic acids or their expression products, peptides, proteins, glycoprotein, carbohydrates, lipids, or glycolipids and mixtures thereof.

In yet another aspect, the present invention provides a method of screening a test compound for the ability of the test compound to alter a phenotype associated with a disease; or to test the effectiveness of a compound believed to be useful in treating a disease or disorder, a compound may be tested by adding the test compound to the medium containing the live teleost. In one embodiment of the present invention, the teleost is contained in an aqueous medium in a microtiter well, such as in a multi-well plate, e.g., a 96-well plate, and test compounds are administered to teleosts by electroporation, lipofection, or ingestion or by using bolistic cell loading technology in which particles coated with the biological molecule are introduced into the cell or tissue of interest as a bolus using a high-pressure gun. Such techniques are well known to those of ordinary skill in the art. See, e.g., Sambrook et al., Molecular Cloning: A Laboratory Manual, 3rd Ed, Peter MacCallum, David Russell, CSHL Press, 2001. This permits an assay to determine the ability of the small molecule to rescue or enhance mutant phenotypes or to disruption or enhance physiological processes in the teleost. Moreover the teleosts may be pretreated prior to exposure to the test compound. Bathing larvae or embryos in fluorescent microbeads used to assay digestive motility is an example of one such pretreatment.

In alternative embodiments, the test compound is administered to the teleost by dissolving the test compound in media containing the fish. Alternatively, the test compound may first be dissolved in the medium and the live teleost submerged in the media subsequently. Such approaches have been used to introduce anesthetics and other chemicals to fish embryos. See, e.g., Westerfield, The Zebrafish Book: A Guide for the Laboratory Use of Zebrafish (3d. ed. 1995)). In yet another alternative, the test compound is administered to the teleost by microinjecting the test compound into the live teleost, or it is administered in conjunction with a carrier. For example, test compounds may be injected into either the yolk or body of a teleost embryo or both.

Test compounds may be administered alone, in conjunction with a variety of solvents (e.g., dimethylsulfoxide (DMSO) or the like) or carriers (including, e.g., peptide, lipid or solvent carriers), or in conjunction with other compounds. For example, the embryo or larvae teleosts are exposed to single or pooled small molecule compounds derived from commercial or NIH sponsored chemical libraries, e.g., NCI Open Synthetic Compound Collection library, Bethesda, Md. Test compounds may be administered to the teleost before, at the same time as, or after administration of a marker used for detection of the response in the animal indicating a specific activity (e.g., cell death activity, angiogenesis activity, toxic activity, cell invasion, organ development, lipid absorption, intestinal motility, fibrosis, bile secretion).

In the methods of the present invention, involving a test compound to be screened for the ability of the compound to alter a phenotype, the method comprises contacting at least one wild type or mutant teleost with a test compound and detecting the teleost in which the phenotype is altered. An exemplified phenotype being altered involved organ structure and/or morphology.

A variety of techniques may be used to detect an alteration in the phenotype. Such techniques, include for example, in situ hybridization, fluorescent labeling, such as by PED6, fluorescent beads, antibody staining of specific proteins, antibody markers that label signaling proteins, and the like. For the lipid absorption screen, more than 3200 random small molecule compounds were examined for their ability to inhibit absorption and processing of the quenched fluorescent lipid, PED-6. These experiments identified 3 compounds (12 hour incubation, 20 uM) that inhibited processing of the fluorescent lipid, as assayed by the absence of intestinal and gallbladder fluorescence normally present in 5 day post-fertilization zebrafish that ingest PED-6. One of these compounds inhibited PED-6 ingestion, demonstrating the effectiveness of the embodied methods of the present invention. Of the 3200 compounds, 67 were easily and rapidly found to be toxic (overnight exposure at 20 uM caused death), thus saving many man-hours of experimentation and needless deaths of test animals. In addition, 15 compounds were identified that selectively inhibited either gallbladder or intestinal fluorescence.

Alterations in phenotype may also be detected by, e.g., visual inspection, colorimetry, fluorescence microscopy, light microscopy, chemiluminescence, digital image analyzing, standard microplate reader techniques, fluorimetry including time-resolved fluorimetry, visual inspection, CCD cameras, video cameras, photographic film, or the use of current or developing instrumentation, such as laser scanning devices, fluorimeters, photodiodes, quantum counters, plate readers, epifluorescence microscopes, scanning microscopes, confocal microscopes, flow cytometers, capillary electrophoresis detectors, or by means for amplifying the signal, such as a photomultiplier tube, etc. Responses may be discriminated and/or analyzed by automated methods using pattern recognition software.

The alterations that are seen in the phenotype depend on the teleost model used, and include any detectable physical or biochemical characteristic of the teleost. The phenotype may be associated with, for example, organ development, protein phosphorylation status, mitotic spindle formation, protein expression, cell morphology, or a disease or disorder in general. The phenotype alteration is considered to be detectable if it may be viewed, observed, determined or recorded by any recognized or developed means: for example, a morphological change, a change in gene expression, or a change in susceptibility to tumor formation. In general, the phenotype change may be observed using various suitable means including microscopy, with or without immunohistochemical staining and RNA-quantification.

Compounds are identified and selected using embodied screening methods of the present invention according to the activities and responses they produce, e.g., as described in the examples that follow that are based upon changes relating to the meltdown (mlt) morphology. Epithelial cysts in homozygous mlt larvae disrupt normal tissue boundaries and occlude the posterior intestinal lumen (see Wallace et al., Developmental Cell 8(5):717-726 (2005b). An expanded layer of connective tissue typical of the desmoplastic reaction seen in many cancers and some benign tumors surrounds most intestinal cysts. These structural defects of the mlt intestine lead to larval death soon after the onset of exogenous feeding.

Histological analyses show that although the intestinal architecture is initially established normally in mlt larvae, it is subsequently disrupted, leading to the formation of massive intestinal cysts. The mlt posterior intestine is comprised of large cysts lined by dysmorphic epithelia, surrounded by expanded connective tissue (Id.) and epithelial invasion is demonstrated in histological analyses. At this stage, both epithelial and mesenchymal cell proliferation are either normal or slightly reduced. These findings suggest that cystic intestinal expansion in mlt mutant larvae does not arise from a primary defect of cell proliferation, but instead is caused by invasion of posterior intestinal epithelial cells into the surrounding stromal tissue.

Automated methods may be readily performed using commercially available automated instrumentation and software, as well as known automated observation and detection procedures. Multi-well formats are particularly attractive for high through-put and automated compound screening. Screening methods may be performed, for example, using a standard microplate well format, with at least one zebrafish embryo in each well of the microplate. This format permits screening assays to be automated using standard microplate procedures and microplate readers to detect alteration of phenotype in the zebrafish embryos in the wells. Microplate readers include any device that is able to read a signal, such as color, fluorescence, luminescence, radioactivity, or shape of the object from a microplate (e.g., 96-well plate).

Methods of detection include fluorometry (standard or time-resolved), luminometry, or photometry in either endpoint or kinetic assays. Using such techniques, the effect of a specific agent on a large number of teleosts (e.g., teleost embryos) may be rapidly ascertained. In addition, with such an arrangement, a wide variety of compounds may be rapidly and efficiently screened for their respective effects on the cells of teleosts contained therein.

Sample handling and detection procedures may be automated using commercially available instrumentation and software systems for rapid reproducible application of markers, dyes and agents, fluid changing, and automated screening of target compounds. To increase the through-put of test compound administration, currently available robotic systems may be used. Such systems include, e.g., BioRobot 9600 (Qiagen Inc., Valencia, Calif.); ZYMATE.RTM (Zymark Corp., Hopkinton, Mass.); and BIOMEK.RTM (Beckman Instruments, Inc., Fullerton, Calif.). Most robotic systems use a multi-well culture plate format.

Automated systems are useful in the processing procedures, which involve a large number of fluid changes required at defined time points. Non-automated through-put is typically about 5 microtiter plates per investigator (assuming 400 teleost embryos and 20 compounds) per week based on using a 96-well plate with 1 embryo per well and screening 2 concentrations with 10 embryos per concentration. However, by using currently available fluid handling hardware (e.g., Bodhan Automation, Inc., Zymark) and standard sample handling procedures, 50-100 plates can be processed per day (4800-9600 teleost embryos and 200-400 compounds). Incorporation of commercially available fluid handling instrumentation significantly reduces the time frame of manual screening procedures and permits efficient analysis of many test compounds, including libraries of agents.

The disease phenotypes contemplated by the methods of the present invention are associated, without intended limitation, with cancer, fibrosis, hematologic disease, immunologic disease, angiogenesis involving embryonic vasculature and many post-natal processes, such as wound healing and tissue and organ regeneration and physiology. Microplate assays can also be used to monitor absorbance, excretion, metabolism or intracellular distribution of a test compound in a teleost. In such methods, the wells provide a means to contain teleosts while a test compound redistributes between the incubation media and the teleosts contained therein, and/or is ingested by or metabolized within the teleost. Initially, the test compound can be in the medium alone, in the teleost alone, or in both the teleost and the medium. After culturing the teleost for a period of time, the amount of the test compound in the medium, or the teleost, or both, is determined. A decrease in the amount of a test compound in the medium over the course of incubation period is a measure of ingestion or absorption of the test compound by the teleost, and allows calculation of an ingestion or absorption rate. An increase in the amount of a test compound in the medium over the incubation time period is a measure of excretion of the agent from the teleost and allows calculation of an excretion rate. By performing the assay with different initial concentrations of test compound in the media and the teleosts, it is possible to calculate the rates of both of these processes. In methods in which the detection assay distinguishes between the test compound and metabolic products of the test compound, it is also possible to calculate a metabolic rate.

Phenotypic Analyses

Mutant phenotypes recovered using the screen of the instant invention can be categorized into several broad categories. First, using morphological and histological criteria mutations that visibly perturb structural development of the pharynx, esophagus, intestine, liver and/or biliary tract are distinguished from those mutants that appear normal. The latter group is considered physiological mutants and is categorized based upon its handling of the panel of fluorescent lipids of the instant invention. This group encompasses, but is not limited to, mutations affecting the intestinal epithelium.

Embryological and transient expression assays also are important studies that can aid phenotypic analyses of zebrafish mutants. Unfortunately, mutations affecting development of the zebrafish digestive organs are, in general, less easily analyzed using these techniques than mutations affecting early development. The short half-life of injected RNA transcripts and DNA expression constructs coupled with the mosaic distribution of the micro-injected DNA limits the utility of transient expression assays for mutations that are not recognizable until 4-5 dpf.

In one embodiment of the instant invention, histological analyses are performed by fixing larvae in 4% paraformaldehyde, embedding the fixed larvae in glycolmethacrylate, and followed by sectioning. Sections are stained using toluene blue/azure II as described and analyzed using a Zeiss Axioplan compound microscope. When needed, selected immunocytochemical and molecular markers are employed to further categorize organ specific defects. If necessary ultrastructural studies are performed as well. For ‘physiological’ mutations, affected larvae are sequentially soaked in the fluorescent lipids of the instant invention, thereby allowing a more detailed categorization.

Test Compounds and High-Through-Put Screening Assays

The invention provides methods for identifying compounds or agents that can be used to treat disorders characterized by (or associated with) aberrant or abnormal physiologic responses resulting from genetic manipulation, and high through-put screening for compounds that rescue the teleosts that are so affected. These methods are also referred to herein as high-through-put screening assays and typically include the step of screening a candidate/test compound or agent for the ability to modulate (e.g., stimulate or inhibit) physiological phenotypic changes. Candidate/test compounds or agents that have one or more of these abilities can be used as drugs to treat disorders characterized by such phenotypic changes and rescue. Candidate/test compounds or agents include, for example, (1) peptides such as soluble peptides, including Ig-tailed fusion peptides and members of random peptide libraries (see e.g., Lam et al., Nature 354:82-84 (1991); Houghten et al., Nature 354:84-86 (1991)) and combinatorial chemistry-derived molecular libraries made of D- and/or L-configuration amino acids; (2) phosphopeptides (e.g., members of random and partially degenerate, directed phosphopeptide libraries, see. e.g., Songyang et al. Cell 72:767-778 (1993)); (3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); and (4) small-organic and inorganic molecules (e.g., molecules obtained from combinatorial and natural product libraries).

In one embodiment, the invention provides a method for identifying a compound capable of use in the treatment of a disorder characterized by (or associated with) aberrant or abnormal phenotypes relating to cancer cell invasion or fibrosis. This method typically includes the step of assaying the ability of the compound or agent to modulate the mutant phenotype in the homozygous larvae, thereby identifying a compound for treating a disorder characterized by the aberrant or abnormal phenotype, such as those set forth herein. Thus, the invention provides high through-put screening assays to identify candidate/test compounds or agents that modulate, for example, but without limitation, cancer cell invasion lipid and/or fibrosis.

Typically, the assays include the steps of identifying at least one phenotypic perturbation, such as the disclosed effect of the mlt mutant on the posterior intestinal epithelial cells of the model zebrafish larvae. At least one quenched or fluorescently-tagged marker is administered to the organism having the phenotypic perturbation, administering a candidate/test compound or agent to the organism under conditions that allow for the uptake of the candidate/test compound or agent by the organism and wherein, but for the presence of the candidate/test compound or agent, the pattern of fluorescence (or lack thereof) would be unchanged. Typically a change in the pattern of fluorescence (indicating “rescue”) is detected by comparing the pattern of fluorescence prior to administration of the candidate/test compound or agent, with that seen following administration of the candidate/test compound or agent.

In the methods of the present invention, a variety of test compounds from various sources may be screened for the ability of the compound to alter a phenotype associated with a disease or disorder to test the effectiveness of a compound believed to be useful in treating a disease or disorder. In accordance with the methods of the present invention, one or more than one test compounds may be screened simultaneously or sequentially. The present invention also includes a compound obtained by the screening methods provided herein.

Test compounds to be screened may be naturally occurring or synthetic molecules or those produced by recombinant technologies. Such naturally-occurring compounds may be obtained from natural sources, such as, marine microorganisms, algae, plants, and fungi, and include minerals or oligo agents. Alternatively, test compounds may be obtained from combinatorial libraries of agents, including peptides or small molecules, or from existing repertories of chemical compounds synthesized in industry, e.g., by the chemical, pharmaceutical, environmental, agricultural, marine, cosmetic, drug, and biotechnological industries. Test compounds may include, e.g., pharmaceuticals, therapeutics, agricultural or industrial agents, environmental pollutants, cosmetics, drugs, organic and inorganic compounds, lipids, glucocorticoids, antibiotics, peptides, proteins, sugars, carbohydrates, chimeric molecules, and combinations thereof.

Combinatorial libraries may be produced for many types of compounds that may be synthesized in a step-by-step fashion. Such test compounds include, without limitation, polypeptides, proteins, nucleic acids, beta-turn mimetics, polysaccharides, phospholipids, hormones, prostaglandins, steroids, aromatic compounds, heterocyclic compounds, benzodiazepines, oligomeric N-substituted glycines and oligocarbamates. In the methods of the present invention, the preferred test compound is a small molecule, nucleic acid and modified nucleic acids, peptide, peptidomimetic, protein, glycoprotein, carbohydrate, lipid, or glycolipid.

Large combinatorial libraries of compounds may be constructed by the encoded synthetic libraries (ESL) method, e.g., as described in Affymax, WO 95/12608, Affymax WO 93/06121, Columbia University, WO 94/08051, Pharmacopeia, WO 95/35503 and Scripps, WO 95/30642 (each of which is incorporated herein by reference in its entirety). Peptide libraries may also be generated by phage display methods, e.g., Devlin, WO 91/18980. Compounds to be screened may also be obtained from governmental or private sources including, e.g., the DIVERSet E library (16, 320 compounds) from ChemBridge Corporation (San Diego, Calif.), the National Cancer Institute's (NCI) Natural Product Repository, Bethesda, Md., the NCI Open Synthetic Compound Collection; Bethesda, Md., NCI's Developmental Therapeutics Program, or the like. Further embodiments of the present invention provide methods of treating a subject in need thereof, including any vertebrate, such as a mammal, more specifically such as human, having a disease or disorder resulting in a phenotype associated with increased or uncontrolled cellular invasion or fibrosis. The method comprises administering to the subject a compound obtained by the screening methods outlined above. A defect phenotype of this type includes, but is not limited to, cancer.

General Experimental Method

Zebrafish, Danio rerio, were housed in a separate facility consisting of approximately 2500 tanks of varying sizes (1 liter, 3.75 liter, and 9 liter). Wildtype and heterozygous mlt adult fish stocks were maintained and crossed as described by Pack et al., 1996 supra, herein incorporated by reference. Environmental conditions were carefully monitored for disease prevention and to maintain fish in perpetual breeding condition. Male and female fish were reared at a density of no more than 8 fish per liter at a constant temperature and light cycle (27-29° C. with the light/dark cycle kept at 14/10 hours) in pre-treated water (heated, charcoal-filtered and UV-sterilized). Fish were fed twice daily with a variety of dried and live foods.

The zebrafish embryos and/or larva were transferred to multi-well plates manually or using an automated dispenser (commercially available). At least a single teleost occupies each well, although in preferred methods of the embodiments exemplified herein, approximately 6-8 embryos were added per well of a 96-well plate using a chemical weighing spatula, although proportionately the number of embryos varies with the size of the well, e.g., ˜15 embryos/well in a 48-well plate (Falcon). In some instances, pre-treatment (as described herein) may be more efficiently conducted in 96 well plates. In alternative methods, the teleosts are synchronized embryos.

For some assays, the embryos and larvae were reared in the wells, meaning that they were placed in the wells long before the assay is performed. In other instances, the embryos and larvae are placed in the wells within 24 hours (or less) of the assay. Because homozygous mlt mutants do not survive to adulthood, there are no adults available from which to obtain further specimens. Consequently, for example, in the present invention germ cells from homozygous mlt embryos are transplanted into wild type fish, and once the presence of the mutation is confirmed, the germ line fish are destroyed. Thus, only the mutant mlt strains survive, which can then be crossed to produce offspring.

It has been previously shown by this inventor and others that PED6 is swallowed by the larvae and transported through the digestive system. Fluorescent cleavage products have been identified in the liver and gall bladder of larvae exposed to PED6, and it provides a clear marker of the intestine, demonstrate that in zebrafish larvae, metabolism of this marker is similar to, if not identical to, other vertebrates.

For testing, the embryos were then cultured in the presence of the test compound(s) overnight or up to 96 hrs at 28.5° C., for example, in 96-well plates. Mutant larvae and mutants reared in the presence of small molecule compounds may be bathed in the fluorescent lipid PED6 or fluorescent microspheres, which outline the digestive tract lumen and can be seen by visual inspection or by a commercially-available fluorescent plate scanner, typically by 96-120 hours post fertilization (hpf). Although signals that regulate regional epithelial differentiation and renewal are poorly understood, in meltdown (mlt), the posterior gut is disorganized and bears an expanded mesenchyme (Briggs, Am. J. Physiol. Regulatory Integrative Comp. Physiol. 282: R3-9 (2002)). Neither fluorescent marker enters the posterior intestine of mlt mutants because the intestinal lumen is obstructed by the massive cysts that form in all mlt mutant larvae.

Positional cloning, chromosomal localization of the mlt locus, sequencing and identification of the myh 11 gene is set forth in detail in Wallace et al., supra, 2005 a & b, herein incorporated by reference in their entirety. Morpholino knockdown experiments (i.e., synthetic molecules used to block access of other molecules to specific sequences within a nucleic acid, thereby knocking down gene function) and sequencing were carried out as set forth in Wallace et al., supra, 2005b, as were mRNA rescue experiments.

Immunohistochemistry, in situ hybridization in 4% paraformaldehye or 2% trichloroacetic acid (TCA) and histological analyses were carried out as set forth in Pack et al., 1996, supra, herein incorporated by reference.

Scoring was, in general, as follows:

No effect: A test compound was considered to have no effect if none of the mlt mutant larvae show fluorescent lipid in the posterior intestine.

Toxic effect: If most of the embryos were dead, delayed, or exhibited some morphologic abnormality, the test compound was considered to be toxic.

Complete rescue: If all embryos had a wild-type or partially rescued phenotype, that test compound was chosen for further analysis. When no mutants were observed, one possibility was that the test compound produced a complete rescue of the mutant phenotype. The other possibility was that there were never any mutants present in the well. When embryos derived from matings of heterozygous mlt/+ fish are used for these assays, then with 8-10 embryos per test compound, the latter possibility may be calculated to occur with a frequency of 0.01%.

Partial rescue: Partial rescue was considered when mutants or the mutant phenotype was present, but the fluorescent markers were present in the posterior intestine.

### Additional Embodiments of the Invention

It will also be appreciated by those skilled in the art that, although certain protected derivatives of the herein-identified compounds, which derivatives may be made prior to a final deprotection stage, may not possess pharmacological activity as such, however, they may be administered parenterally or orally and thereafter metabolized in the body to form compounds of the invention which are pharmacologically active. Such derivatives are, therefore, described as “prodrugs.” All such prodrugs are included within the scope of the present invention.

The invention further encompasses compounds which are structurally similar to the herein-identified compounds, e.g., structural analogs, or derivatives thereof. Preferably, a derivative has at least 75%, 85%, 95%, 99% or 100% of the biological activity of the reference compound. In some cases, the biological activity of the derivative may exceed the level of activity of the reference compound. Derivatives may also possess characteristics or activities not possessed by the reference compound. For example, a derivative may have reduced toxicity, prolonged clinical half-life, or improved ability to cross the blood-brain barrier. Such prodrugs, derivatives or similar compounds are encompassed within the terms “test compounds” or “herein-identified compounds.”

The methods disclosed herein provide for the parenteral or oral administration of the identified compound to a subject, such as a human, in need of such treatment. Parenteral administration includes, but is not limited to, intravenous (IV), intramuscular (IM), subcutaneous (SC), intraperitoneal (IP), intranasal, and inhalant routes. In the method of the present invention, the compound is preferably administered orally. IV, IM, SC, and IP administration may be by bolus or infusion, and may also be achieved by a slow release implantable device, including, but not limited to pumps, slow release formulations, and mechanical devices. The formulation, route and method of administration, and dosage will depend on the disorder to be treated and the medical history of the patient. For parenteral or oral administration, compositions of the compound may be semi-solid or liquid preparations, such as liquids, suspensions, and the like.

The invention further provides a pharmaceutical composition comprising a compound obtained using the present invention or as set forth herein. Preferred compositions comprise, in addition to the compound, a pharmaceutically acceptable carrier (i.e., sterile and non-toxic) liquid, semisolid, or solid diluent that serves as a pharmaceutical vehicle, excipient, or medium. Any diluent known in the art may be used. Exemplary diluents include, but are not limited to, water, saline solutions, polyoxyethylene sorbitan monolaurate, magnesium stearate, methyl- and propylhydroxybenzoate, talc, alginates, starches, lactose, sucrose, dextrose, sorbitol, mannitol, glycerol, calcium phosphate, mineral oil, and cocoa butter. Suitable carriers or diluents are described, for example, in Remington: The Science and Practice of Pharmacy, by A R Gennaro, ed. A L Gennaro, Lippincott, Williams & Wilkins; ISBN: 0683306472; 20th edition, 2000, a standard reference text in this field, which is incorporated herein by reference in its entirety. Preferred examples of such carriers or diluents include, but are not limited to, water, saline, Ringer's solution, dextrose solution, and 5% human serum albumin. Liposomes and nonaqueous vehicles, such as fixed oils may also be used. The formulations are sterilized by commonly used techniques.

The compositions, or pharmaceutical compositions, comprising the nucleic acid molecules, vectors, polypeptides, antibodies and compounds identified by the screening methods described herein, may be prepared for any route of administration including, but not limited to, oral, intravenous, cutaneous, subcutaneous, nasal, intramuscular or intraperitoneal. The nature of the carrier or other ingredients will depend on the specific route of administration and particular embodiment of the invention to be administered. Examples of techniques and protocols that are useful in this context are provided herein.

The dosage of these compounds will depend on the disease state or condition to be treated and other clinical factors, such as weight and condition of the human or animal and the route of administration of the compound. For treating human or animals, between approximately 0.25 μg/kg of body weight to 100 mg/kg of body weight of the compound can be administered. Therapy is typically administered at lower dosages and is continued until the desired therapeutic outcome is observed.

The invention also provides an article of manufacture (a “kit”) comprising packaging material and a pharmaceutical composition contained within said packaging material, wherein said packaging material comprises a label which indicates said pharmaceutical may be administered, for a sufficient term at an effective dose, for evaluating metabolic processes or for treating and/or preventing, e.g., cancer, hematologic disease, immunologic disease, angiogenesis defects involving embryonic vasculature and many post-natal processes, such as wound healing and tissue and organ regeneration, solid tumor growth, and pathogenic components in numerous diseases, including rheumatoid arthritis, atherosclerosis, diabetes mellitus, retinopathies, psoriasis, and retrolental fibroplasias, bone diseases, cardiovascular disease, obesity and cholesterol deposits, neurodegenerative disease or metabolic disorders and the like. It is also used for the study of metabolic processes in a vertebrate, such as mammal, including a human, wherein the pharmaceutical composition comprises a compound obtained using the present invention or as set forth herein.

The present invention is further illustrated by the following examples, which should in no way be construed as being further limiting. Fish mutations discussed in the specification, as well as mutants representing new model diseases, can be created using the methods set forth herein.

### EXAMPLES

### Example 1

Identifying Small Molecule Compounds that Inhibit Cancer Cell Invasion and/or Fibrosis.

To identify genes and pharmacological compounds that regulate cancer progression using the zebrafish, meltdown (mlt) larvae was used. Mlt is a recessive lethal mutation previously selected by mutagenesis screening that is displayed as an altered phenotype of intestinal architecture. The disruption in the zebrafish larva results in cystic expansion of the posterior intestine as a result of stromal invasion of nontransformed epithelial cells (Farber et al., 2001, supra).

In the present assays, mosaic zebrafish were generated in which homozygous mlt/mlt cells have replaced the native germline of wild type fish. 100% of the offspring derived from pair wise matings of such modified zebrafish are homozygous for the mlt mutation. Thus, all of the larvae placed in each well of the 96-well plate are mlt mutants, greatly enhancing the efficiency of each assay. Note that it is also possible to perform assays using larvae derived from non-mosaic, heterozygous mlt/+ fish. However, using this approach, only 25% of the larvae in each well would display the recessive mlt phenotype.

Mutant larvae derived from the aforementioned matings are arrayed in the 96-well plates at ˜60 hours post-fertilization (hpf). Chemical compounds are added at this time. At 96 hpf, the quenched fluorescent phospholipid N-((6-(2,4-dinitrophenyl)amino)hexanoyl)-1-palmitoyl-2-BODIPY-FL-pentanoyl-sn-glycero-3-phosphoethanolamine (PED6) (Farber et al., 2001, supra) or a related fluorescent compound were added to the wells. At 120 hpf, the larvae were imaged via fluorescent microscopy to determine whether fluorescent lipid is present in the posterior intestine of the mutant larvae. Non-rescued larvae lack such fluorescence, whereas it is present in rescued larvae (confirmed using a TGF-β inhibitor known to block small molecule activity, thereby rescuing the mutant phenotype.) Details of this example, including the results and conclusions drawn are presented in detail in Wallace et al., supra, 2005a & b, attached hereto and incorporated in their entirety for all purposes.

Positional Cloning. Chromosomal localization of the mlt locus was performed via bulk segregant analysis using zebrafish potentially polymorphic simple sequence repeat (SSR) markers randomly distributed on each zebrafish linkage group. For fine mapping of the mlt locus, a chromosomal walk originating with bacterial artificial chromosome (BAC) clones spanning markers on either side of the mlt locus (z7647 and kldf gene) was performed. Two overlapping BAC clones spanning the mlt locus were ultimately identified (127N6 & 192P17; zebrafish RPCI-71 BAC library).

Polymorphisms within BAC ends were identified by heteroduplex analysis. One and three larvae were identified that were recombinant for polymorphisms in the 127n06 and 192p17 BAC ends, respectively (2500 embryos). Zero of 2500 mutant embryos were recombinant for a polymorphic marker (derived from the Sp6 end of BAC 161n07) within the myh11 gene.

Shotgun sequencing of two overlapping BAC clones identified sequences corresponding to only three genes: myh11, the UBE2G2 gene, a ubiquitin-conjugating enzyme ortholog, and a gene orthologous to the predicted human gene FLJ31153 that is located adjacent to human MYH11 on chromosome 16. Further analyses of genomic sequence available from the zebrafish Ensemble assembly identified a predicted gene with low sequence homology to mammalian low-affinity nerve growth factor receptor, but no other genes (not shown). The two critical region BACs did not contain sequences corresponding to this putative receptor homolog. Full-length predicted myh11 cDNA sequence derived from the BAC clones was confirmed using RT-PCR with RNA derived from adult and larval zebrafish. The mlt mutation in the myh11 gene was located 13, 420 by from the zero recombinant marker derived from BAC 161n07. This marker was located within the myh11 gene.

Rescued mlt larvae and 48 hpf mlt embryos were identified molecularly. The dCAPs finder 2.0 program (http://helix.wustl.edu/dcaps/dcaps.html) was used to design PCR primers that introduced an mboI restriction site into a 300 by genomic DNA fragment from the MYH11^{mlt} allele. Restriction digestion of DNA fragments amplified from MYH11^{mlt} and wild-type MYH11 alleles using the dCAPS primers generated 270 by and 300 by fragments, respectively. Primers used for genotyping are identified in Wallace et al. supra, 2005b, and are herein incorporated by reference in their entirety.

To clone zebrafish β4 integrin cDNA, ESTs with homology to the human β4 integrin gene were identified (fm94e01 and fm84d11). Full-length sequence was obtained using 5′ and 3′ RACE protocols using RNA derived from 5 dpf larvae.

Morpholino Knockdowns. All morpholinos were injected into 1- to 4-cell stage fertilized embryos. Injection of 620 pg of a morpholino directed against the 5′region of the zebrafish smooth muscle myosin heavy chain cDNA that overlapped the predicted translation initiation site completely rescued all mlt mutants. Injection of lower doses generated partially rescued mlt mutants. Rescued mutants were identified molecularly.

For MT-mmpa, morpholinos directed against either the 5′ region of the MT-mmpa, cDNA or an intron-exon boundary within the catalytic domain of MT-mmpa, were used (injected dose: 1.2 ng). For β4 integrin, a morpholino directed against an intron-exon boundary within the cytoplasmic domain was used (injected dose: 410 pg). Intron-exon splice acceptor morpholinos were designed using genomic contigs identified in the zebrafish Ensemble database.

For rescue experiments, 1-cell stage embryos derived from pair-wise mating of heterozygous mlt carriers were injected with either the MT-mmpa, morpholino alone or in combination with the β4 integrin morpholino. Subsequently, all embryos were injected with either 0.375 or 0.1875 pg of tissue inhibitor of metalloproteinases 2 (TIMP2 inhibitor) (PF021; Oncogene Research Products, now Calbiochem, San Diego, Calif.) at 48 hpf. Alternatively, 2.5 dpf or 3 dpf larvae were incubated in the TGF-β Type I receptor inhibitor SB 431542, 100 μM in embryo media. Morpholino sequences were as identified in Wallace et al., supra, 2005b for smooth muscle myosin heavy chain, β4 integrin, and MT-mmpa.

Immunohistochemistry, In Situ Hybridization, and Histology. Embryos were fixed in either 4% paraformaldehyde or 2% trichloroacetic acid (TCA) (Sigma, Milwaukee, Wis.) for 2 hours at room temperature. Histological analyses and in situ hybridization experiments were performed as described by Pack et al., supra, 1996. Whole mount in situ specimens processed for histology were counterstained with Nuclear Fast Red. For immunohistochemistry, embryos were pretreated with 160 ng/μl collagenase (Sigma) for 10 min. Primary antibody was incubated overnight at 4° C. Embryos were washed 3× in PBS containing 0.2% Tween (PBST), and secondary antibody was incubated for 2 hr at RT and then washed in PBST. Embryos were embedded in JB-4 plastic (Polysciences, Inc., Warrington, Pa.) and sectioned by microtome (5 μm). Sections were imaged using confocal (Zeiss LSM 510) or fluorescent microscopy.

Primary antibodies are rabbit anti-laminin (1:100 dilution) (Sigma); rabbit anti-desmin (1:100 dilution) (Sigma); rabbit anti-β4 integrin (1:100 dilution) (Sigma); rabbit anti-smooth muscle myosin (1:100 dilution) (Biomedical Technologies); and mouse anti-ZO1 (1:50 dilution). Secondary antibodies are Alexa Fluor 488 conjugated anti-rabbit and Alexa Fluor 568 conjugated anti-mouse (Molecular Probes, Eugene, Oreg.).

30 mM BrdU was injected into the yolk of the embryo. One hour later, embryos were fixed in 4% paraformaldehyde for 2 hr at room temperature or overnight at 4° C. Fixed embryos were digested with proteinase K (Roche Diagnostics, Mannheim, GE; 745 723) (10 to 20 ng/ml) for 30 min. The embryos were incubated in 2N HCL for 1 hr. Incorporated BrdU was detected with an anti-BrdU antibody (Roche) and visualized with Peroxidase substrate kit (Vector Laboratories, Burlingame, Calif.). For quantification of cell proliferation, histological sections through the posterior intestine of whole-mount specimens that had been counterstained with nuclear fast red were analyzed.

Construction of W512R mutant chicken smooth muscle myosin cDNA, generation of myosin protein in baculovirus-SF9 cells, and ATPase assays of phosphorylated HMM-like fragments were performed as described (Sweeney et al., J. Biol. Chem. 273:6262-6270 (1998)).

mRNA Rescue Experiments. Full-length wild-type and W512R mutant myh11 cDNAs were cloned into pCS2. Sense strand mRNA was transcribed (Ambion Message Machine) and injected into 1-cell stage mlt and heterozygous mlt/+ embryos. t the highest dose injected, 30% of 24 hpf larvae had nonspecific developmental defects.

Results

The recessive lethal mlt mutation causes cystic expansion of the larval zebrafish posterior intestine, but epithelia of the anterior intestine and other organs are unaffected in mlt mutants, mlt larvae develop normally. Epithelial cysts in homozygous mlt larvae disrupt normal tissue boundaries and occlude the posterior intestinal lumen. An expanded layer of connective tissue typical of the desmoplastic reaction seen in many cancers and some benign tumors surrounds most intestinal cysts. These structural defects of the mlt intestine lead to death soon after the onset of exogenous feeding. Histological analyses show that the mlt posterior intestine is comprised of large cysts lined by dysmorphic epithelia surrounded by expanded connective tissue. In contrast, the wild-type posterior intestine is organized as a simple epithelial tube lined by columnar epithelial cells. At 74 hours postfertilization (hpf), when the mlt phenotype is first recognizable, ruffling of the posterior intestine is visible in live larvae. At this stage, both epithelial and mesenchymal cell proliferation are either normal or slightly reduced, meaning that cystic intestinal expansion in mlt mutant larvae does not arise from a primary defect of cell proliferation.

Histological analyses of early mlt mutants (74 hpf) revealed additional findings that provided an explanation for the development of posterior intestinal cysts. In all mlt larvae examined (n=5), focal regions of stratified epithelia were identified in the posterior intestine. Intestinal structure in the intervening regions separating these focal disruptions was normal. Immunohistochemical analyses revealed basement membrane irregularities or frank disruption in the abnormal regions of all mlt mutants, with invasion of individual or contiguous epithelial cells in these affected regions. Importantly, a normal pattern of laminin encircling the basal surface of polarized epithelial cells within the mlt intestine was present before the mutant phenotype was recognizable (55 hpf). Together, these data show that intestinal architecture is initially established normally in mlt larvae, but is subsequently disrupted, leading to the formation of massive intestinal cysts.

Bulk segregant analysis placed the mlt locus on zebrafish chromosome 6. High-resolution meiotic mapping identified a genomic contig spanning the mlt locus that contained zebrafish orthologs of three human genes. Sequencing of cDNAs for all three genes derived from wild-type and mlt larvae identified a single base substitution in only the myh11 gene. This T to C transition led to a substitution of arginine for tryptophan 512, a conserved amino acid in the rigid relay loop of all vertebrate myh11 genes. These data show that the mlt phenotype does not arise from a loss of myosin motor function. Knockdown of smooth muscle myosin protein in mlt larvae confirmed this hypothesis. Microinjection of an antisense morpholino that targets the translation initiation site of zebrafish myh11 rescued mlt larvae in a dose-dependent manner Rescued mlt larvae were morphologically indistinguishable from wild-type siblings, but do not survive to adult stages. Interestingly, recurrent intestinal cysts were noted in 20% of rescued mlt larvae (n=80) at 16 dpf, confirming myh11 as the responsible mlt gene and show that the altered myosin protein induces an invasive phenotype of both developing and mature intestinal epithelial cells.

Studies showed protein biochemical analyses using an orthologous chicken smooth muscle myosin protein, engineered to harbor an identical W512R amino acid substitution, functioned as a constitutively active (e.g., hypermorphic) ATPase that was active with or without phosphorylation of the regulatory light chain, which regulates contraction of wild-type myosin. In addition, the phosphorylated and dephosphorylated mutant myosin had 8- to 10-fold greater ATPase activity than wild-type myosin in the absence of actin and no motor function in an in vitro motility assay (not shown). These results are consistent with the effects of mutations in orthologous regions of Dictyostelium myosin II that abolish myosin motor function and also elevate basal ATPase activity. However, loss of myosin motor function cannot account for the mlt phenotype, since antisense knockdown of myosin protein in wild-type larvae does not produce a mlt phenocopy.

Taken together, these data support a model in which the mlt mutation alters epithelial architecture in a cell-nonautonomous fashion by interfering with the integrity of cells identified as smooth muscle and, as a result, stromal-epithelial cell signaling. This model is consistent with intestinal myh11 expression that is restricted to stromal cells in larval zebrafish and other vertebrates. Thus, epithelial invasion in mlt mutants results from either the loss of a signal that normally maintains epithelial architecture or the production of a signal that causes intestinal epithelial cells to adopt an invasive phenotype. Since heterozygous mlt/+ larvae do not develop intestinal cysts, smooth muscle cells appear sensitive to the dosage of the mutant myh11 allele.

To further define the nonautonomous nature of the mlt phenotype, the expression of genes known to play a role in cancer cell invasion was analyzed. The membrane-type metalloproteinase-1 (MT1-mmp) and metalloproteinase-2 (mmp2) genes are commonly implicated in invasive human cancers. Zebrafish mlt mutants ectopically express the MT1-mmp (mmp14a) and mmp2 orthologs within the posterior intestinal epithelium. Gene knockdowns of mmp14a gave early lethal phenotypes, however, partial knockdown of mmp14a coupled with injection of the mmp2 inhibitor TIMP2 partially rescued mlt larvae (see below). Compared with mock-injected mlt mutants, rescued mlt larvae had a discernable lumen in regions of the posterior intestine, a phenotypic variation never seen in uninjected mlt mutants. Immunostainings of rescued mlt larvae revealed localized intestinal regions with normal architecture that lacked desmin+smooth muscle cells.

Normal epithelial architecture in these segments of rescued larvae supports the finding that epithelial invasion in mlt mutants does not arise from disruption of a physical barrier, but instead from altered smooth muscle signaling that activates epithelial metalloproteinases and other proinvasion genes.

Further testing was performed to determine whether other genes implicated in epithelial invasion were activated in mlt mutants. Elevated α6β4 integrin expression is reported in human tumors and cancer cells, and increased mount RNA signaling through the β4 integrin subunit has been implicated in cancer cell survival and invasion. mlt mutant larvae ectopically express immunoreactive β4 integrin in intestinal regions where epithelial architecture is perturbed. Partial knockdown of a zebrafish β4 integrin ortholog using a morpholino designed to truncate the cytoplasmic domain of the β4 integrin protein that plays a role in adhesion and cell signaling did not rescue the mlt phenotype. However, co-injection of the MT-mmpa and β4 morpholinos, with the TIMP2 peptide, rescued a higher percentage of mlt mutant larvae (54.6% of 108 mlt larvae) compared with MT-mmpa knockdown and TIMP2 injection (11.6% of 120 mlt larvae).

Activation of the TGF-β signaling pathway has also been implicated in cancer cell invasion. TGF-β signaling in cancer cells may be activated through either autocrine (autonomous) or paracrine (nonautonomous) mechanisms. TGF-β signaling also appears to play a role in epithelial invasion in mlt mutants. Dpf mlt mutants were observed to ectopically express the TGF-β1 gene within the intestinal epithelium, as well as the TGF-β target genes snail1 and snail2, which have been shown to downregulate expression of E-cadherin in invasive and migratory mammalian epithelial cells. Mutant larvae (5 dpf) treated with a small molecule inhibitor of the mammalian TGF-β Type 1 receptor at 2.5 dpf or 3 dpf had far fewer cysts than untreated mlt larvae, suggesting that TGF-β signaling regulates progression of the mlt phenotype. These data, together with the results of metalloproteinase and integrin inhibition experiments, suggest that common molecular pathways regulate the invasive phenotype of human cancers and mlt intestinal epithelial cells.

Thus, it was determined that epithelial invasion in mlt mutants is regulated by the ectopic expression of genes causally linked to human cancer progression. Inhibition of these cancer progression genes thus rescued the mlt mutants, meaning that the single amino acid mutation is responsible for disrupting the smooth muscle integrity around the zebrafish intestine. As a result, when the mlt mutation was positionally cloned to the smooth muscle myosin heavy chain myh11, it was determined that the mlt mutation constitutively activates the Myh11 ATPase, which disrupts smooth muscle cells surrounding the posterior intestine. The gain-of-function myh11 allele encodes a myosin protein that functions as a constitutively active ATPase and lacks motor function. Immunohistochemical and ultrastructural analyses showed that the mutant myosin selectively disrupts posterior smooth muscle cells, which in turn causes basement membrane disruption, epithelial invasion, and ultimately, cystic intestinal expansion.

Adjacent epithelial cells ectopically express metalloproteinases, integrins, and other genes implicated in human cancer cell invasion. Knockdown and pharmacological inhibition of these genes restores intestinal structure in mlt mutants, despite persistent smooth muscle defects. Accordingly, these data identify an essential role for smooth muscle signaling in the maintenance of epithelial architecture and support gene expression analyses and other studies that identify a role for stromal genes in cancer cell invasion. Normal intestinal development of the heterozygous mlt/+ fish indicates that gene dosage is important in the development of the mutant phenotype, but spatial restriction of the epithelial defects to the posterior intestine was an unexpected feature of the mlt phenotype.

The knockdown experiments showed that constitutive activity of the Myh11 protein, rather than the loss of myosin function, accounts for the mlt epithelial defect. Knockdown of the mutant myosin protein rescued mlt larvae, but the juvenile rescued fish do not survive to adult stages. Because the present immunohistochemical assays showed the rescue effect on myh11 translation to be transient, the juvenile fish survive with a defective, nonfunctional smooth muscle myosin protein.

Substantial evidence was found that supports the use of mlt mutants to model cancer cell invasion. Alterations in the tissue architecture seen in the mlt intestine are characteristic of invasive cancers, including human cancers. Epithelial invasion, which is a prominent aspect of the mlt phenotype, is a hallmark of cancer and is never seen in benign tumors unless they have undergone transformation. No evidence was seen of regulated epithelial remodeling during normal zebrafish development (Wallace et al., Devel. Biol. 255:12-29 (2003)). The mlt mutants ectopically expressed orthologues of human genes that regulate cancer invasion, and genetic and pharmacological targeting of these genes restores the intestinal structure of the mlt mutants. Furthermore, these factors indicate the importance of developing high-throughput screens to identify regulators of cancer cell invasion in zebrafish.

In a related assay, transgenic mlt larvae are engineered that express a fluorescent reporter of gene whose expression is enhanced in human fibrotic diseases. Examples of such genes include colAl (Type I collagen) or the hsp-47 gene, a collagen chaperone that is up-regulated in the intestine of MLT mutants (not previously disclosed—not published). These transgenic larvae allow screening for small molecules that inhibit fibrosis. Related reporter transgenes may be engineered using the regulatory elements of other genes that are up-regulated in the mlt intestine. These genes appear to be directly relevant to the mlt invasion phenotype. These included (not previously disclosed) AP-1 family genes, such as c-jun and c-fos (and other AP-1 family members).

There is growing recognition that cancer may become a chronic disease. If treatments are long term, the toxicity profile of drugs, which can be examined readily in the transparent teleost (e.g., zebrafish) larvae, will become an increasingly important parameter for drug screening and evaluation. The foregoing data suggest that human MYH11 polymorphisms could, in theory, predispose primary cancers to develop an invasive phenotype. Such mutations or polymorphisms would selectively influence the function of cancer-associated stromal cells, but not normal tissue stroma. The finding that a single base pair mutation in zebrafish myh11 generates an invasive phenotype of epithelial cells within the posterior, but not anterior, zebrafish larval intestine is supportive of such selective stromal cell susceptibility.

High-Throughput Analyses in mlt Mutants. An advantage of a model organism, such as the zebrafish, is the ability to do relatively high through-put forward genetic and pharmacologic screens. Modifier screens for suppressors or enhancers of the mlt phenotype, using either bioactive small molecules, which have already been successfully used in zebrafish, or classical mutagenesis strategies may help identify novel suppressors of genes that regulate cancer cell invasion.

### Example 2

Identification of Small Molecule Compounds that Enhance Intestinal Motility.

Zebrafish sparse mutants (c-kit mutation) have delayed intestinal transit. This defect arises from a lack of intestinal pacemaker cells known as interstitial cells of cajal (ICC) (unpublished data). Delayed transit may be assayed through the persistence of ingested fluorescent microbeads in the intestine of sparse mutant larvae. However, sparse is a non-lethal mutation in homozygotes.

For this assay, 96 hpf viable sparse mutant larvae derived from matings of homozygous sparse/sparse adult fish are arrayed in 96 well plates. The larvae are bathed in fluorescently labeled microbeads (commercially available) for ˜8 hours. Because the sparse mutants do not expel the labeled beads, one of ordinary skill in the art practicing this invention can readily determine whether a compound has been administered to the mutant larvae that alters that phenotype—so that the fluorescent compounds begin to be expelled from the larvae in a timely manner. The larvae are then exposed to small molecule compounds for ˜16 hours. At 120 hpf, fluorescent microscopy (as described above) are used to identify compounds that “rescue” the sparse mutant larvae, thereby enabling it to eliminate the fluorescent beads from its intestine. Note that sparse mutants are available through the zebrafish stock center (ZIRC—http:/zfin.org/zirc/home/guide.php).

### Example 3

High Through-Put Screening to Identify Seizure Inhibiting Compounds.

Based upon the finding that stereotypic and concentration-dependent seizures can be elicited by exposure to a common convulsant agent (pentylenetetrazole, PTZ) in a simple vertebrate system e.g. zebrafish larvae (Baraban et al., Neuroscience 131:759-768 (2005)), the methods described above were applied to demonstrate their effectiveness in high through-put screening for compounds to treat a disease affecting a different system, the CNS. However, the methods used were completely different from those described by Baraban et al., in that the zebrafish larvae were selected at a different stage of development, and the present invention would not have operated on the immobilized assays described therein, but the observed seizure activity permitted the principle to be applied to further confirm the breadth of the capability of the present invention.

The fish or more specifically, the larva are treated with the test compounds prior to onset of the seizure, at the time of seizure onset, or after the seizures have been induced.

To assay small molecule compounds with anti-seizure activity, 4 day post-fertilization wild type zebrafish were placed in 96-well plates (5 larvae per well) and incubated in the test compounds (20 uM) as above, for either 2 or 12 hrs. Subsequently, the larvae were administered pentylenetetrazole (PTZ; 15 uM) and observed for seizure activity (i.e., a change in phenotype). Wild type zebrafish larvae exhibit seizure activity, manifest as increased locomotion or rapid circular movements, within 5 to 10 minutes of exposure to PTZ. This activity persists for approximately 20 minutes and then culminates in a tonic convulsion that abolishes movement and normal posture. Prior to or after onset of the seizure activity, the small molecule test compound was administered to the larvae and the response of the fish assessed visually or by digital recording.

Scoring was based upon whether the test compound was able to inhibit or modify the seizure activity of the larvae. However, given the relatively brief duration of the seizure activity, it was found to be more effective to analyze the larvae in half of the wells of one 96-well plate before adding PTZ to the remaining wells of the 96-well plate. Accordingly, chemically induced seizures in zebrafish can be inhibited in a concentration-dependent fashion, demonstrating the effectiveness of the high through-put screening methods of the present invention for rapidly determining chemical treatments epilepsy or genetic modifiers of seizure disorders, or even for identifying compounds to prevent the onset of seizure in a patient with a seizure disorder like epilepsy.

The disclosures of each patent, patent application and publication cited or described in this document are hereby incorporated herein by reference, in their entirety.

While the foregoing specification has been described with regard to certain preferred embodiments, and many details have been set forth for the purpose of illustration, it will be apparent to those skilled in the art without departing from the spirit and scope of the invention, that the invention may be subject to various modifications and additional embodiments, and that certain of the details described herein may be varied considerably without departing from the basic principles of the invention. Such modifications and additional embodiments are also intended to fall within the scope of the appended claims.

1-36. (canceled)

37. A method for high through-put screening for physiologic alterations in an altered teleost displaying a phenotype that is characteristic of the alteration and different from a wild-type, unaltered, matched teleost, comprising the steps of:
contacting the teleost displaying a genetically-inherited or chemically-induced phenotype with at least one test compound for a sufficient time and under suitable conditions to induce a response in the teleost indicative of pharmacological activity of the compound;
introducing a labeled reagent to the contacted teleost and test compound under conditions that allow for uptake of the reagent by the teleost, wherein binding of the labeled reagent to, or with, the teleost generates a detectable signal dependent upon and characteristic of the teleost's response;
detecting the signal and comparing the response from that of a matched control teleost that was not contacted with the test compound or the labeled reagent, wherein a change in the teleost signal that is different from that of the control, indicates an altered phenotype and pharmacological activity of the at least one test compound; and
outputting a report of same.

38. The method of claim 37, wherein the altered phenotype is associated with or representative of a disease selected from the group consisting of: cancer, hematologic disease, immunologic disease, angiogenesis, rheumatoid arthritis, atherosclerosis, cardiovascular disease, obesity and cholesterol deposits, mellitus, retinopathies, psoriasis, bone diseases, liver diseases, and retrolental fibroplasias, neurodegenerative disease and metabolic disorders, or wherein the phenotype is useful for studying metabolic processes.

39. The method of claim 37, wherein the teleost is a zebrafish.

40. The method of claim 37, wherein the teleost is an embryo, larva or adult.

41. The method of claim 39, wherein the teleost is a zebrafish embryo or larva.

42. The method of claim 37, wherein the teleost is contained in a microtiter well.

43. The method of claim 37, further comprising homogeneously distributing the test compound in media containing the teleost.

44. The method of claim 37, further comprising providing a labeling reagent to the at least one test compound in a form that is ingestible by the teleost.

45. The method of claim 44, wherein the labeling reagent is fluorescent.

46. The method of claim 45, wherein the fluorescent label is a lipid, peptide or lipoprotein.

47. The method of claim 37, wherein the at least one test compound is selected from the group consisting of a small molecule, nucleic acid, peptide, protein, glycoprotein, carbohydrate, lipid, and glycolipid.

48. The method of claim 47, wherein the at least one test compound is a small molecule.

49. The method of claim 37, further comprising selecting the teleost from among mutants having a particular phenotype or from among modified mutants that facilitate high through-put screening, or from among transgenic teleosts having a particular phenotype or those displaying at least one organ-specific visible marker.

50. A compound obtained by the method of claim 37.

51. A zebrafish having an altered phenotype resulting from treatment in accordance with the method of claim 37, wherein the alteration indicates activity of the test compound.

52. A method for high through-put screening of a test compound for the ability of the compound to alter a genetically altered teleost displaying a phenotype that is characteristic of the alteration and different from a wild-type, unaltered, matched teleost, comprising the steps of:
contacting the teleost displaying a genetically inherited or chemically-induced phenotype with at least one test compound for a sufficient time and under suitable conditions to induce a response in the teleost indicative of pharmacological activity of the compound;
introducing a labeled reagent to the contacted teleost and test compound under conditions that allow for uptake of the reagent by the teleost, wherein binding of the labeled reagent to, or with, the teleost generates a detectable signal dependent upon and characteristic of the teleost's response;
detecting the signal and comparing it to the response from a matched control teleost that was not contacted with the test compound or the labeled reagent, wherein a change in the teleost signal that is different from that of the control, indicates an altered phenotype and pharmacological activity of the at least one test compound; and
outputting a report of same.

53. The method of claim 52, wherein the altered phenotype is associated with a disease, selected from the group consisting of cancer, hematologic disease, immunologic disease, angiogenesis, rheumatoid arthritis, atherosclerosis, cardiovascular disease, obesity and cholesterol deposits, mellitus, retinopathies, psoriasis, bone diseases and retrolental fibroplasias, neurodegenerative disease and metabolic disorders, or wherein the phenotype is useful for studying metabolic processes.

54. The method of claim 52, wherein the teleost is a zebrafish.

55. The method of claim 52, wherein the teleost is an embryo, larva or adult.

56. The method of claim 54, wherein the teleost is a zebrafish embryo or larva.

57. The method of claim 52, wherein the teleost is contained in a microtiter well.

58. The method of claim 52, further comprising homogeneously distributing the test compound in media containing the teleost.

59. The method of claim 52, further comprising providing a labeled reagent to the at least one test compound in a form that is ingestible by the teleost.

60. The method of claim 59, wherein the labeled reagent is fluorescent.

61. The method of claim 60, wherein the fluorescent label is a lipid, peptide or lipoprotein.

62. The method of claim 52, wherein the at least one test compound is selected from the group consisting of a small molecule, nucleic acid, peptide, protein, glycoprotein, carbohydrate, lipid, and glycolipid.

63. The method of claim 52, wherein the at least one test compound is a small molecule.

64. The method of claim 52, further comprising selecting the teleost from among mutants having a particular phenotype, or from among modified mutants that facilitate high through-put screening, or from among transgenic teleosts having a particular phenotype or those displaying at least one organ-specific visible marker.

65. A compound obtained by the method claim 52.

66. A zebrafish having an altered phenotype resulting from treatment in accordance with the method of claim 52, wherein the alteration indicates activity of the test compound.

67. The method of claim 52, further comprising identifying an agent(s) to prophylacticly or therapeutically treat a disease or disorder characterized by uncontrolled cellular invasion.

68. The method of claim 67, wherein the disease or disorder comprises cancer or fibrosis.

69. A method of treating a host having, or susceptible to, a disease or disorder characterized by uncontrolled cellular invasion or fibrosis, said method comprising administering a test compound selected by the methods of claim 52, wherein the labeled reagent is pharmaceutically acceptable.

70. The methods of claim 52, further comprising identifying a gene(s) involved in the regulation of cellular invasion.

71. The method of claim 70, wherein cellular invasion comprises cancer or fibrosis.

72. A kit comprising packaging material and a plurality of altered teleosts displaying a phenotype that is characteristic of the alteration and different from a wild-type, unaltered, matched teleost, together with a pharmaceutically acceptable marker, wherein the packaging material comprises a label or instruction sheet, which indicates uses of the contents of the kit for high through-put screening for a composition causing physiologic alterations in the phenotype of the altered teleost.
